(19) United States
(12) Patent Application Publication
(10) Pub. No.: US 2015/0047634 A1
(43) Pub. Date: Feb. 19, 2015

(54) DEVICE FOR DELIVERING HYDROGEN TO A SUBJECT

(71) Applicant: UNIVERSITY HEALTH NETWORK, Toronto (CA)

(72) Inventors: Kieran Murphy, Toronto (CA); David Mikulis, Toronto (CA); Joseph Barfett, Toronto (CA)

(21) Appl. No.: 14/385,725

(22) PCT Filed: Mar. 18, 2013
(86) PCT No.: PCT/CA2013/000261

§ 371 Date: Sep. 16, 2014

Related U.S. Application Data
(60) Provisional application No. 61/612,262, filed on Mar. 17, 2012.

(51) Int. Cl. A61M 16/12; A61M 16/22; A61M 16/10; A61M 16/20; A61M 16/00; A61M 16/06

(52) U.S. Cl. CPC A61M 16/12; A61M 16/0003; A61M 16/0045; A61M 16/0688; A61M 16/1075; A61M 16/208; A61M 16/22; A61M 16/0078; A61M 2016/0027; A61M 2016/0033; A61M 2205/3334; A61M 2205/3368; A61M 2202/02
USPC 128/202.26; 128/203.28; 128/203.14

(57) ABSTRACT

There is provided herein devices for delivering hydrogen to a subject comprising a flow path including a breather bag and an inhalation outlet for engagement with the subject respiratory system; and a breathing gas supply source comprising hydrogen gas and in fluid communication with the flow path. Also provided are devices for the cutaneous delivery of hydrogen.

### CROSS-REFERENCE TO RELATED APPLICATIONS

This application claims priority to U.S. Provisional Application No. 61/612262, filed on Mar. 12, 2012, the entirety of which is hereby incorporated by reference.

### TECHNICAL FIELD

The present disclosure relates to the field of medical devices, and more particularly to the field of gas delivery devices.

### BACKGROUND

Ionizing radiation (IR) is known to exert a deleterious genetic effect on an irradiated organism due to the interaction of high energy photons with water to generate free radicals. These free radicals, hydroxyl (OH) radicals in particular, have potential to interact with and damage DNA. In the context of medical imaging exams, genetic damage caused by IR may manifest as cancer many years after the exposure event. As one means of their activity, free radical scavenging agents (i.e. antioxidants) are thought to reduce the genetic damage caused by IR by neutralizing free radicals before they interact with genetic material.

In order for a free radical scavenger to be effective, however, it must first transverse the nucleus and accumulate around DNA. Numerous oral and potentially intravenous antioxidants have been described as radio-protective agents in a literature that dates back to the cold war.

Areas in which medical device technology still needs to develop include delivery systems for antioxidants.

### SUMMARY

In various aspects, the present disclosure provides devices, systems, uses, and methods of delivering antioxidants to a subject.

In accordance with one aspect of the present disclosure, there is provided a device for delivering hydrogen to a subject. The device includes: a flow path including a breather bag and an inhalation outlet for engagement with the subject respiratory system; and a breathing gas supply source comprising hydrogen gas and in fluid communication with the flow path.

In accordance with another aspect of the present disclosure, there is provided a device for cutaneous delivery of hydrogen. The device includes: a patch comprising an engagement layer for engagement with a subject's skin; and an inlet for delivery of hydrogen gas into the patch under pressure. The engagement layer is in fluid communication with the inlet and permits the diffusion of hydrogen into the subject's skin.

In accordance with another aspect of the present disclosure, there is provided use of a device for radioprotection of a subject. In one example, the device includes: a flow path including a breather bag and an inhalation outlet for engagement with the subject respiratory system; and a breathing gas supply source comprising hydrogen gas and in fluid communication with the flow path. In another example, the device includes: a patch comprising an engagement layer for engagement with a subject's skin; and an inlet for delivery of hydrogen gas into the patch under pressure, wherein the engagement layer is in fluid communication with the inlet and permits the diffusion of hydrogen into the subject's skin.

Further details of these and other aspects of the described embodiments will be apparent from the present disclosure.

### BRIEF DESCRIPTION OF THE DRAWINGS

Embodiments of the present invention are described by way of example with reference to the attached figures, wherein:

FIGS. 1 and 2 show side schematic views of example devices in subject respiratory systems for delivering hydrogen to a subject.

FIGS. 3 and 4 show cross-sectional views of example devices for cutaneous delivery of hydrogen.

### DETAILED DESCRIPTION

Gaseous antioxidants, such as hydrogen gas, can, in some examples, have advantages over chemical antioxidants as radio-protective agents due to their potentially rapid administration before and during radiation exposure, non-toxicity, ease of membrane passage and therefore attainment of a significant nuclear concentration, and simplicity of clinical implementation as there is no need to fill prescriptions or take oral or IV medications.

In some examples, delivery of such gaseous antioxidant agents can be optimized so as to create an increased local concentration of hydrogen within the tissues under the greatest IR exposure or, alternatively, to increase delivery of such gases to tissues which are of the greatest radiosensitivity and therefore at highest risk of future malignancy. Such tissues in particular can include the gastrointestinal, oral and esophageal mucosa, skin, the bone marrow and in the context of cataract formation, the lens of the eye.

Further, a major difference between diagnostic and therapeutic radiation is that the former typically does not cause an upregulation of DNA repair while the latter does. The same can be said of an acute exposure (seconds to minutes) versus a chronic exposure (hours to days), where only the latter will tend to upregulate cellular repair processes.

A person at high altitude will be exposed to more cosmic rays and the body upregulates DNA repair to deal with the added burden. If a person receives radiation therapy, there is such marked free radical formation that DNA repair is upregulated.

Diagnostic imaging is relatively low dose and occurs nearly instantly. As such the body does not respond to the DNA damage that occurs. This leads to the potential for DNA strand breaks to go undetected by cellular repair processes and result in oncogenesis. Thus in the setting of diagnostic radiation, particularly with short term exposures, radiation protection becomes uniquely important.

The practical implication of antioxidant gases can be applied to medical devices which, in some examples, can improve ease of use for patients and their care-takers, safety of implementation, or affordability.

There are several antioxidant gases available in the medical setting. The most commonly used gases include anesthetic agents, xenon in particular. There has been substantial literature generated on the antioxidant gas Edaravone, which has also been associated with some toxicity.

Hydrogen gas is a recently described powerful antioxidant that, in some examples, has been shown to have radioprotective properties. Hydrogen can be stored in a compressed form, bound to various alloys, or generated through chemical reaction or the electrolysis of water.

Hydrogen has several properties which distinguish it from other gases. Firstly, hydrogen is of low molecular weight and hence is exceptionally diffusive. Hydrogen can dissolve into metals, and will readily cross tissues and cell membranes. Hydrogen is thus ideal for rapid delivery to cells in the medical imaging context.

A major drawback to the use of hydrogen is flammability. Hydrogen gas is flammable in room air at concentrations above 4%. To some extent the propensity for combustion is linked to gas temperature and oxygen concentration. Thus hydrogen concentrated in air above a certain threshold will also be non-flammable. Typically this point is described as greater than 90% in air.

Dangerous gases are rarely used in the medical environment and generally only with appropriate safety measures. Specific new devices are described herein which in some embodiments may have improved safety profiles. If the hydrogen level is maintained below the flammable range of approximately 4%, then administration of the gas can be generally considered to be safe. Concentrations above 4% can be used transiently and then generally, only with safety features in place which can limit exposure to flame or static discharge. With appropriate safety measures in place, hydrogen can be administered in a generally safe manner.

Examples of routes of hydrogen delivery in the medical context can include inhaled hydrogen, trans-cutaneous delivery of hydrogen, and enteric delivery of hydrogen.

As most radiation exposures in a medical imaging context occur over a few seconds, in some examples, a patient's blood hydrogen level can be maintained for a relatively short time. Capacity for transient increased hydrogen delivery can, in some examples, be built into these devices.

Inhalation of hydrogen can in some examples provide a simple means of delivering the gas to patients during medical imaging exams. Because hydrogen is flammable and readily generated by electrolysis, in some examples, hydrogen for individual imaging exams can be generated. In some examples, this can be safer than storing large quantities on hydrogen in or around the imaging area.

In some examples of water electrolysis, the amount of hydrogen generated can be controlled via the amount of water in the device performing electrolysis to limit the potential for exceeding the flammable limit of hydrogen in the ambient air surrounding a patient and medical staff. Similarly, in some examples, small hydrogen storage systems are available, for example using compressed gases or by solid state storage in alloys, either of which can provide controlled release of small quantities of the therapeutic gas.

Although it can be possible to maintain the hydrogen concentration in a scan room within a range below 4%, in some examples, breathing devices applied to the face can be adapted to existing work flows.

Hydrogen gas, by virtue of its low molecular weight, also has propensity to rise in any sealed chamber and hence could collect at the ceiling of a scan room and present a potential flammability hazard. A similar situation has been met with Xenon in the context of nuclear medicine exams where filters are used to collect and dispose of the dense xenon gas at the floor. In some embodiments, the devices described herein can implement hydrogen gas as a radioprotective agent and in some examples can cause little or minimal disruption to existing work flows and can be applied directly to patients.

Breathing apparatuses can lend themselves well to the delivery of hydrogen gas through inhalation, and can be variations of anesthetic machines. In some examples, hydrogen enriched air can be provided to a patient by means of face mask, nasal prong, nasal tent, laryngeal mask, or a variety of other means. Where a concentration of hydrogen is below 4%, there can be little or no risk of flammability in these systems in room air.

Re-breathers can, in some examples, be employed to maintain a patient's hydrogen level using only minimal quantities of the gas. A variety of example rebreathing circuits are described.

In some examples, a rebreather can use a relatively small quantity of hydrogen while sustaining reinhalation during a medical imaging exam. For example, in a system where a patient breaths into and out of a paper bag through their mouth, where a 4% hydrogen level could be sustained in the bag, only a scant quantity of the dangerous gas can be needed.

In one example embodiment, taking into account dead space in the patient's lungs and aerodigestive tract, and a 1-2 L rebreather bag volume, approximately 20 cc's of hydrogen gas would be needed to maintain a 4% level. In some examples, less than 250 cc's would be needed, which is a relatively small volume with a small flammability hazard.

In some examples, this amount of hydrogen can be generated for each exam on an individual basis without need to store larger quantities of the gas, either through electrolysis or through a small storage system which can be coupled to a rebreather.

An example rebreather for this application can have features including a CO₂ absorber or other CO₂ sink, an oxygen input which may or may not be computer controlled, as well as a variety of gas and pressure detectors and a suction system to dispose of the dangerous gases.

In some examples, permanent rebreather systems can be used as an alternative to disposable rebreathers. In some examples, these systems can be more complex, and in some examples, can offer a fine degree of control of blood gases including hydrogen, oxygen, and carbon dioxide. In some examples, a technologist running a medical imaging device can precisely control a patient's inhaled/exhaled hydrogen concentration. In some examples, this concentration may be maintained below 4%. In some examples, this concentration may also be transiently elevated above 4% shortly before an exam and flushed shortly after the exam.

In some examples, permanent rebreathers can be more complex and can, in some examples, require specialized skills to operate. In some examples, the controls for such a rebreather can be integrated into a scanning system to more precisely deliver increased hydrogen levels immediately before an exam. In some examples, medical imaging technologists operate devices from behind radioprotective leaded glass. Although a rebreathing system could be positioned in a scan room in contact with a patient, controlling such a system from a computer controller could require reconfiguration of such systems.

In some examples, disposable devices can be simpler for patients and technologists to use and/or implement than a permanent rebreather. In some examples, they can be implemented without any significant changes to existing technology or work flow. In some examples, disposable systems could offer relatively less control over blood gases.

Most CT scan rooms are equipped with suction. This is particularly useful in settings where a patient's airway is compromised by debris or secretions which are sucked away by an operator. These suction lines are rarely used.

Oxygen is also widely available in medical imaging exam rooms. Oxygen is used more routinely in the medical imaging context.

In some example embodiments, the devices can make use of suction and oxygen as it is available in the medical imaging context. In the case of hydrogen delivery, in some example devices, suction can be used to remove excess gas which might pose a flammability risk. In the case of oxygen, some example devices include face masks designed to accommodate an oxygen line to supplement a patient's air if need be.

A permanent hydrogen re-breathing system can be a device that in some examples can be permanently or semi-permanently associated with a medical imaging scanner system. In the context of CT, an example system could be integrated into the scanner itself or exist beside the scanner. In the context of mammography, an example unit can be positioned beside a patient or integrated into the mammography scanner.

An example system can be controlled along with the scanner from a control panel which is typically behind lead glass, or can be activated by a technologist while still in the scan room and then can be monitored and operated autonomously via a suitable computer controller while the technologist exits to the protected environment.

With a system that can be activated and left alone for the duration of the scan, in some examples, there can be no need to integrate controls which are operated from a protective environment. However, in some such systems, it is possible that a particular hydrogen level must be chosen a priori and cannot be manipulated during the exam. In some examples, it may be desirable for a technologist to transiently elevate an inhaled hydrogen level moments before a scan up to or exceeding the flammability range, and then promptly decrease the hydrogen level back to a safe range immediately after the exam.

In some embodiments, a large hydrogen storage system with such a rebreather can be provided. In some examples, the system can include a tank of compressed hydrogen or hydrogen enriched gas mixture. In some examples, hydrogen can be generated by electrolysis for individual exams. In some of these embodiments, the amount of water or energy available for electrolysis can be controlled to generate only a sufficient quantity of hydrogen.

The scan room can preferably be ventilated to manage minor quantities of escaped gas.

Choosing a specific rebreathing circuit would be within the knowledge of a person skilled in the art. In some examples, the circuit can include elements such as a determined quantity of gas that is re-breathed, ports into this gas pool to provide supplemental oxygen, hydrogen or suction. In some examples, the circuit can be equipped with appropriate gas detectors and/or computer controllers to maintain a desired preset level of antioxidant gas.

The flammability limit of hydrogen can, to some extent, be influenced by temperature and oxygen saturation. Cooler temperatures and lower oxygen levels reduce the potential for hydrogen combustion. In some examples, the rebreathed air in a circuit can be optionally cooled to further mitigate the possibility of combustion, or can be maintained under reduced oxygen tension or pressure.

In appropriate conditions, any of the example devices described herein can be optionally low density and radiolucent and made from materials immune to static discharge and possibly grounded.

In some examples, these devices can be used to induce a desired level of hypoxia, which is itself radioprotective.

### Rebreathing System

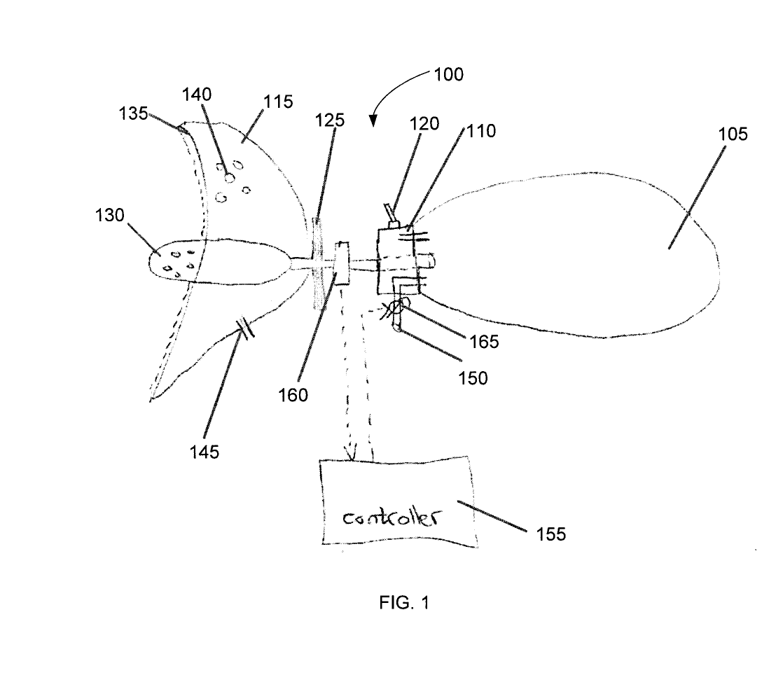

FIG. 1 illustrates an example device 100 for delivering hydrogen to a subject. In some examples, the device is part of a subject respiratory system. In some examples, the subject respiratory system can be a disposable rebreathing system.

In some examples, the device includes a flow path for providing hydrogen to a subject. As shown in the example device 100 in FIG. 1, the flow path can include a reservoir 105. In some examples, the reservoir can be a breather bag such as a rebreather bag. The flow path can also include an inhalation outlet for engagement with the subject respiratory system.

In some examples, the flow path includes means of connecting the reservoir 105 to a patient's face, nose or mouth.

The subject respiratory system can, in some examples, include a breathing gas supply source in fluid communication with the flow path. In some examples, the subject respiratory system can include a hydrogen generator 110. In the example device in FIG. 1, the hydrogen generator 110 is incorporated with a re-breathing bag 105 and a mask 115. In some examples, the hydrogen generation can be activated by a switch 120 or valve on the device 100. In some examples, the subject respiratory system can be a contained disposable system.

In some examples, the device 100 or system can include a carbon dioxide (CO₂) absorber 125. In some examples the device can include a disposable breathing bag 105 and mask 115 which can be attached to a more permanent hydrogen source.

In the example in FIG. 1, the disposable rebreathing system includes a hydrogen generator 110 coupled to a breather bag 105 and face mask 115. In some examples, the generator 110 can be disposable and can create a quantity of hydrogen suitable for an individual exam. In some examples, the generator can create hydrogen for a period of approximately 10 minutes which in some examples, can be suited for CT (computed tomography) or mammography.

In some examples, hydrogen release can be activated by a switch/valve 120 on the device 100. In some examples, a mouthpiece 130 can be connected to the breather bag 105. In some examples, the rebreathing can be achieved through the patient's mouth by means of the mouthpiece 130.

As illustrated in FIG. 1, an optional CO₂ absorber 125 can be connected in series between the mouthpiece 130 and the breather bag 105. In some examples, a CO₂ absorber can help to maintain a patient's blood CO₂ level during an exam. In some examples, the patient's nose can remain open to facilitate breathing should the patient feel the need to receive supplemental air or oxygen. This can, in some examples, provide the patient with a degree of control over their own hydrogen delivery and the option of breathing through their nose should any problem or discomfort with the device occur.

In some examples, the system is further coupled to a mask 115 which can cover a patient's nose and mouth. In some examples, the mask 115 can be tightly adhered to the face via an adhesive tape or adhesive surface 135 which may be integrated with the device. In some examples, the adhesive surface 135 can be around the edge of the mask 115. The mask 115, in some examples, has perforations 140 which can permit the flow of room air. In some examples, the mask 115 can extend to cover the breather bag 105.

In some examples, the mask can include a connection point 145 for connecting to suction which can be turned on during use. In some examples, as a patient is rebreathing through their mouth, this can cause room air to be constantly passed over the face and suctioned away to ensure the safe disposal of any leaked gas. In some examples, at the conclusion of the exam, the patient can inhale gas from the rebreather through his/her mouth and exhale through his/her nose. In some examples, gas including any excess hydrogen exhaled through the nose can be suctioned away via the connection point 145.

In some examples, the mask 115 and/or breather bag 105 can optionally include a connection point 150 for connecting with an oxygen or air source.

A system such as the example subject respiratory system can in some examples provide a transient elevation of hydrogen concentration into a flammable range. In some examples, additional safety features can be incorporated into the device. For example, in addition to the disposable system as described above, a hydrogen detector can be associated with the device to monitor hydrogen levels in the breathing bag. In some examples, if elevation in the flammable range is detected for more than a predetermined period of time, (for example, 1-2 seconds), the contents of the bag can be automatically drained to suction or diluted via the connection point 145, connection point 150, or otherwise.

In some examples, the device 100 can optionally include a control system for controlling a level of hydrogen in the device 100 and/or delivered to the subject. In some examples, the control system can maintain a level of hydrogen at about 4% or less.

In some examples, the control system can include a controller 155 and a hydrogen sensor 160. In some examples, the controller 155 can receive a signal from the hydrogen sensor 160 indicating a level of hydrogen.

In some examples, the controller 155 can control a valve 165 or other device for controlling the input of oxygen or air via connection point 150. In some examples, the controller 155 can be configured to dilute the contents of the breather bag 105 when the hydrogen level is above a threshold level.

In some examples, the controller 155 can be configured to flush the system with oxygen or air when a flammable level of gas is detected.

In some examples, the breather bag 105 its connection with the mouthpiece 130 can be contained within a second bag which can also be optionally connected to suction.

### Non-Rebreathing System

In some examples, a device can provide hydrogenated air to a patient during a scan without using a rebreathing system. In some examples, a non-rebreathing system can be used when hydrogen levels below the flammable range of approximately 4% are used.

Commonly, CT and mammography exams can occur transiently with tissue radiation occupying a few seconds up to a few minutes in time. In some examples, a source of air/hydrogen (optionally with oxygen enrichment) can be administered to patients using face masks or masks. In some examples, the masks can be associated with a breathing bag (i.e. a non-rebreathing bag). In some examples, associating a breathing bag with a medical mask can improve gas delivery.

In some examples, a system can promote inhalation through the patient's mouth and exhalation through the patient's nose. In some examples, the system can promote inhalation through the nose and exhalation through the nose. The exhaled gas can, in some examples, be vented through suction.

In some examples, a disposable rebreather system, such as the example system described above, can be modified such that gas can be infused into the breathing bag continuously, and can be inhaled be the patient through the mouthpiece, and exhaled through the patient's nose which is under a facemask under suction.

In some examples, the system can include a mask within a mask with suction maintaining room air in the potential space between the two masks. In some examples, suction can be used to help adhere the mask to the patient's face.

The system can, in some examples, have a hydrogen source which is uncoupled from an air/oxygen input. In some examples, the hydrogen source can be associated with the bag or mouthpiece, and hydrogen can be eluted into the inhaled air flow path at a controlled rate.

In some examples, the hydrogen source can be disposable. In some examples, the hydrogen source can be connected to the mask apparatus. In some examples, the hydrogen source and a connected mask apparatus can be disposable and can, in some examples, prevent contamination between patients.

In some examples, only the mask apparatus is disposable.

In some examples, the system can include control systems and can include gas detectors.

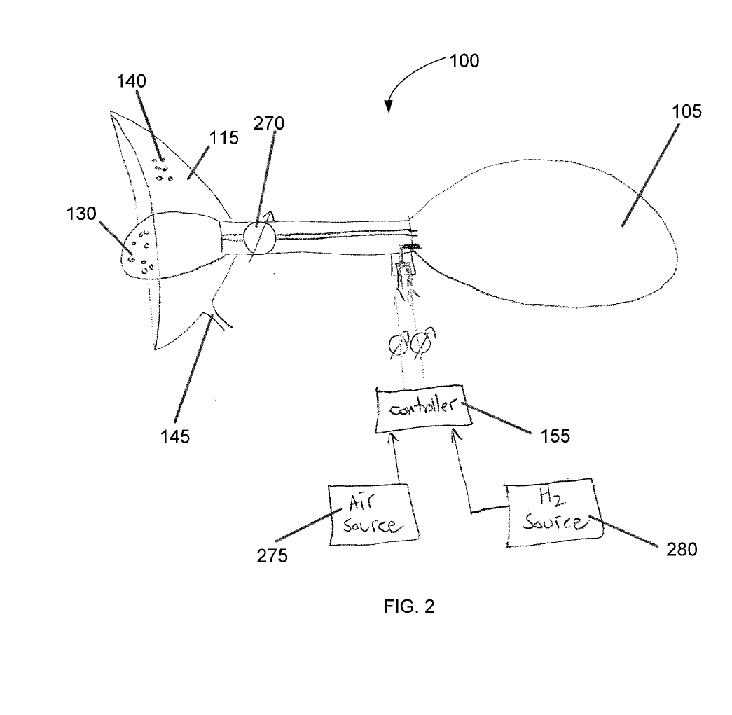

An example non-rebreather system including a device 100 for delivering hydrogen to a subject is illustrated in FIG. 2. The system includes a reservoir such as a breathing bag 105. In some examples, the breathing bag 105 can be sized based on the size or other attribute of the patient. In some examples, the bag 105 can be flexible.

The bag 105 can, in some examples, be connected to the mouthpiece 130 via a one-way valve 270. In some examples, the one-way valve 270 can permit the bag 105 to refill between breaths.

Similar to the examples described above, the flow path can be associated with a mask 115 with perforations 140 and a connection point 145 for connecting suction.

In some examples, all or any subset of these components can be disposable.

The system can, in some examples, include an air source 275 and a hydrogen source 280. In some examples, the air source can provide oxygenated air, and in some examples, can include an optional oxygen enrichment means.

In some examples, such as the system in FIG. 2, the air source 275 and the hydrogen source 280 can both feed into the breathing bag 105.

The relative rates of gas infusion can be defined a priori by a user. In some examples, a controller 155 can be configured to control the relative rates of gas infusion.

In a use example, a patient can inhale the gas mixture from the mouthpiece 130, emptying some of all of the gas in the bag 105. The patient can then exhale through the nose where the exhaled gas can be collected by a vacuum or suction via connecting point 145. In some examples, exhalation through the mouth can be prevented or reduced by the one-way valve 270, and can in some examples, be deferred to the suction system via a release valve.

In some examples, flow rates into the bag 105 are adjusted via a pressure gauge to refill the bag during the exhalation. In some examples, the pressure gauge can be included in or connected to the controller. The controller 155 can, in some examples, be configured to inflate the bag 105 with a defined pressure and a defined ratio of gases. In some examples, the controller 155 can be configured such that the pressure does not exceed the limit of the one-way valve 270.

In some examples, the system can include a patient activatable switch which can be activated during inhalation or exhalation to close a valve connecting the mouthpiece 130 and the bag 105.

In some examples, the system can include flow meters for monitoring the direction of breathing and the controllers.

In some examples, a controller system can include a temperature controller for controlling the temperature of the gas in the device and/or delivered to the subject.

### Cutaneous Delivery System

By virtue of being of such low molecular weight and hence diffusive, in some examples, hydrogen gas can be delivered transdermally.

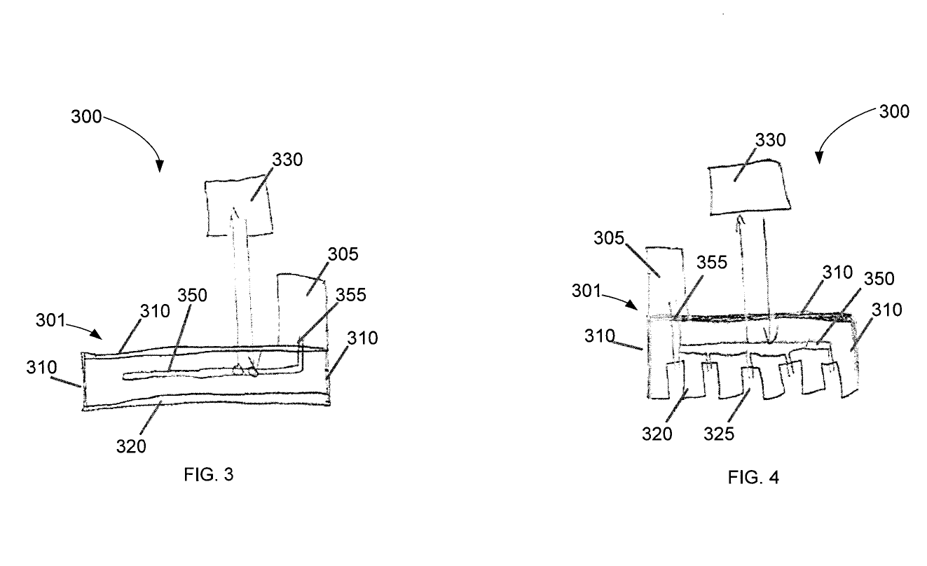

FIGS. 3 and 4 illustrate example devices 300 for transdermal or cutaneous delivery.

In some examples, a system can provide pressurized hydrogen gas adjacent to the skin. The system can, in some examples, create a pressurized hydrogen chamber adjacent to the skin contained under a patch 301. The patch 301 can include an engagement layer 320 for engagement with a subject's skin. The engagement layer 320 can, in some examples, be hydrogen permeable.

In some examples, the patch can comprise an adhesive bandage. The patch can be various sizes. Initial experimentation has shown that a 2″×2″ bandage is an option.

In some examples, the device 300 can be kept at low density and can be radiolucent.

The device can include an inlet 355 for delivery of hydrogen gas into the patch under pressure. The inlet 355 can be in fluid communication with the engagement layer 320 and can permit the diffusion of hydrogen into the subject's skin.

In some examples, the patch can include an outlet in fluid communication with the inlet 355 for irrigating gas. In some examples, the device can include channels 350 in fluid communication with the inlet and/or the engagement layer 320.

The system can include a source of hydrogen in fluid communication with the inlet 355, such as, for example, hydrogen generator 305. The hydrogen generator 305 can generate hydrogen using electrolysis, or in any manner such as those described herein or as otherwise known to a skilled person.

In some examples, only a small amount of hydrogen gas can be required for an application. Even under pressure, the quantity of hydrogen kept adjacent to the skin can be small. In some examples, the hydrogen generated 305 can be an electrolysis cell which can, in some examples, be disposable, incorporated into the bandage and/or radiolucent.

In some examples, the device 300 can be associated with compressed hydrogen, stored hydrogen or can use a connected electrolysis unit which can be continuous with or otherwise associated with the device 300.

The external surfaces 310 of the device can, in some examples, be hydrogen impermeable.

In some examples, the engagement layer 320 can have a fully adhesive undersurface. In some examples the engagement layer 320 can have an adhesive perimeter.

In some examples, the engagement layer 320 can be porous.

With reference to FIG. 3 and/or FIG. 4, in some examples, the engagement layer can include microchannels to facilitate hydrogen delivery.

With reference to FIG. 4, in some examples, the engagement layer 320 can include one or more gas cells 325. In some examples, the borders of the gas cells 325 can be adhesive and the one or more gas cells 325 can be formed to contain pressurized gas.

In some examples, the devices can permit greater levels of adhesion, which can, in some examples, permit higher juxtacutaneous hydrogen pressures.

In some example systems, the generated gas, cells, or porous material can be heated or cooled to facilitate a particular application. For example, the system can optionally include a gas heater or cooler 330.

In some examples, where transdermal hydrogen delivery is to be used for systemic hydrogen administration, the system can heat the skin which can encourage cutaneous vasodilation, which in turn can hydrogen absorption. In some examples, the coupling of temperature modulation can promote venous drainage of the hydrogen and hence systemic circulation.

In some examples, where the application is to protect skin from radiation damage, or to protect subcutaneous muscle and fat from radiation damage, the system or devices can similarly cool the skin. In some examples, cooling can reduce the cutaneous blood volume by causing vasoconstriction, hence; a higher hydrogen level is maintained as a function of depth in skin. In some examples, this can facilitate hydrogen diffusion into subcutaneous tissues which can be at high risk of radiation induced injury. In some examples, cooling and vasoconstriction can promote hypoxia which is radioprotective.

In some examples, the device can include a controller for controlling the temperature of gas in the patch 301.

Any of the example devices described herein can be modified for different applications. For example, a large device can, in some instances, provide hydrogen delivery to those in need of free radical scavenging including patients undergoing medical imaging exams, burn victims, and patients of any age who are otherwise sick.

In some examples, devices to protect the skin can be modified as appropriate to protect the breasts during radiotherapy, the mucosa of the head and neck, the rectum and perineum during prostate radiotherapy, etc.

In some examples, any of the example devices described herein can be used for radioprotection of a subject. In some examples, the use can be for radioprotection against diagnostic levels of radiation.

Although preferred embodiments of the invention have been described herein, it will be understood by those skilled in the art that variations may be made thereto without departing from the spirit of the invention or the scope of the appended claims. All documents disclosed herein are incorporated by reference.

### REFERENCES

1) Cardinal J S, Zhan J, Wang Y, Sugimoto R, Tsung A, McCurry K R, Billiar T R, Nakao A. Oral hydrogen water prevents chronic allograft nephropathy in rats. Kidney Int. 2010 Jan;77(2):101-9. Epub 2009 Nov. 11.
2) Hosgood S A, Nicholson M L. Hydrogen sulphide ameliorates ischaemia-reperfusion injury in an experimental model of non-heart-beating donor kidney transplantation. Br J Surg. 2010 February;97(2):202-9.
3) Mishra P K, Tyagi N, Sen U, Givvimani S, Tyagi S C. H2S ameliorates oxidative and proteolytic stresses and protects the heart against adverse remodeling in chronic heart failure. Am J Physiol Heart Circ Physiol. 2010 February;298(2):H451-6. Epub 2009 Nov. 20.
4) Kimura Y, Goto Y, Kimura H. Hydrogen sulfide increases glutathione production and suppresses oxidative stress in mitochondria. Antioxid Redox Signal. 2010 January;12(1):1-13.
5) Oharazawa H, Igarashi T, Yokota T, Fujii H, Suzuki H, Machide M, Takahashi H, Ohta S, Ohsawa I. Protection of the retina by rapid diffusion of hydrogen: administration of hydrogen-loaded eye drops in retinal ischemia-reperfusion injury. Invest Ophthalmol Vis Sci. 2010 Jan;51(1):487-92. Epub 2009 Oct. 15.
6) Fujita K, Seike T, Yutsudo N, Ohno M, Yamada H, Yamaguchi H, Sakumi K, Yamakawa Y, Kido MA, Takaki A, Katafuchi T, Tanaka Y, Nakabeppu Y, Noda M. Hydrogen in drinking water reduces dopaminergic neuronal loss in the 1-methyl-4-phenyl-1,2,3,6-tetrahydropyridine mouse model of Parkinson's disease. PLoS One. 2009 Sep. 30;4(9):e7247.
7) Chen C, Chen Q, Mao Y, Xu S, Xia C, Shi X, Zhang J H, Yuan H, Sun X. Hydrogen-Rich Saline Protects Against Spinal Cord Injury in Rats. Neurochem Res. 2010 Mar. 31. [Epub ahead of print]
8) Nakao A, Toyoda Y, Sharma P, Evans M, Guthrie N. Effectiveness of hydrogen rich water on antioxidant status of subjects with potential metabolic syndrome-an open label pilot study. J Clin Biochem Nutr. 2010 March;46(2):140-9. Epub 2010 Feb. 24.
9) Qian L, Cao F, Cui J, Huang Y, Zhou X, Liu S, Cai J. Radioprotective effect of hydrogen in cultured cells and mice. Free Radic Res. 2010 March;44(3):275-82.
10) Liu C, Cui J, Sun Q, Cai J. Hydrogen therapy may be an effective and specific novel treatment for acute radiation syndrome. Med Hypotheses. 2010 Jan;74(1):145-6. Epub 2009 Aug. 6.
11) Nakao A, Sugimoto R, Billiar T R, McCurry K R. Therapeutic antioxidant medical gas. J Clin Biochem Nutr. 2009 January;44(1):1-13. Epub 2008 Dec 27.
12) Buchholz B M, Kaczorowski D J, Sugimoto R, Yang R, Wang Y, Billiar T R, McCurry K R, Bauer A J, Nakao A.
13) Buchholz B M, Kaczorowski D J, Sugimoto R, Yang R, Wang Y, Billiar T R, McCurry K R, Bauer A J, Nakao A. Hydrogen inhalation ameliorates oxidative stress in transplantation induced intestinal graft injury. Am J Transplant. 2008 October;8(10):2015-24. Epub 2008 Aug. 22.
14) Ohsawa I, Ishikawa M, Takahashi K, Watanabe M, Nishimaki K, Yamagata K, Katsura K, Katayama Y, Asoh S, Ohta S. Hydrogen acts as a therapeutic antioxidant by selectively reducing cytotoxic oxygen radicals. Nat Med. 2007 June;13(6):688-94. Epub 2007 May 7.

1. A device for delivering hydrogen to a subject comprising:
a. a flow path including a breather bag and an inhalation outlet for engagement with the subject respiratory system; and
b. a breathing gas supply source comprising hydrogen gas and in fluid communication with the flow path.

2. The device of claim 1, further comprising a controller system for controlling a level of hydrogen in the device and/or delivered to the subject.

3. The device of claim 2, wherein the level of hydrogen is maintained at about 4% or less.

4. The device of claim 2, wherein the controller system comprises a hydrogen sensor.

5. The device of claim 2, wherein the controller system further controls a level of oxygen in the device and/or delivered to the subject.

6. The device of claim 2, wherein the controller system further comprises an oxygen sensor.

7. The device of claim 2, wherein the controller system further controls a level of CO₂ in the device and/or delivered to the subject.

8. The device of claim 2, wherein the controller system further comprises a CO₂sensor.

9. The device of claim 1, wherein the breathing gas supply source generates hydrogen using electrolysis.

10. The device of claim 1, further comprising a CO₂ absorber disposed along the flow path.

11. The device of claim 1, further comprising a suction disposed along the flow path for venting excess gas and/or exhaled gas.

12. The device of claim 1, further comprising a source of oxygen in fluid communication with the flow path.

13. The device of claim 1, wherein the inhalation outlet comprises a nose and mouth mask.

14. The device of claim 1, wherein the inhalation outlet comprises a mouthpiece.

15. The device of claim 1, wherein the controller system comprises a temperature controller for controlling the temperature of the gas in the device and/or delivered to the subject.

16. The device of claim 1, wherein the controller system comprises a pressure controller for controlling the pressure of the gas in the device and/or delivered to the subject.

17. The device of claim 1, wherein the flow path does not allow exhaled gas into the breather bag.

18. The device of claim 1, wherein the breather bag is a rebreather bag and the flow path allows the flow of exhaled gas into the rebreather bag.

19-31. (canceled)
